(19) United States
(12) Patent Application Publication
(10) Pub. No.: US 2016/0096865 A1
(43) Pub. Date: Apr. 7, 2016

(54) Peptide-Silica Hybrid Materials

(71) Applicants: Centre National de la Recherche Scientique (CNRS), Paris (FR); Universite Montpellier 2 Sciences et Techniques, Montpellier (FR); Universite of Montpellier 1, Montpellier (FR)

(72) Inventors: Jean MARTINEZ, Caux (FR); Gilles SUBRA, Juvignac (FR); Ahmad MEHDI, Montpellier (FR); Said JEBORS, Montpellier (FR); Christine ENJALBAL, Les Matelles (FR); Luc BRUNEL, Montpellier (FR); Francois FAJULA, Teyran (FR)

(73) Assignee: Universite de Montpellier I, Montpellier (FR)

(21) Appl. No.: 14/410,293

(22) PCT Filed: Jun. 24, 2013
(86) PCT No.: PCT/EP2013/063171

§ 371 Date: Oct. 13, 2015

(30) Foreign Application Priority Data
Jun. 22, 2012 (FR) 1255962

(51) Int. Cl. C07K 1/04; A01N 55/00; C07C 45/72

(52) U.S. Cl. CPC C07K 1/042; C07C 45/72; A01N 55/00

(57) ABSTRACT

The invention relates to novel peptide-silane “hybrid block” molecules, to the synthesis thereof and to the use of same for producing novel peptide-silica hybrid materials that can be used in various applications.

The subject matter of the present patent application relates to novel peptide silane “hybrid block” molecules, to the synthesis thereof and to the use of same for preparing novel peptide-silica hybrid materials which can be used in various applications, for example in medical equipment, in separating complex products, or in nanoparticles for imaging.

Generally, silica-based organic-inorganic hybrid materials obtained by sol-gel to processes have attracted considerable attention in recent decades. Indeed, these materials constitute a fascinating class of products which combine properties of organic fragments on inorganic matrices (Loy, D. A. et al., Chem. rev. 95, 1431-1442 (1995) and Corriu, Angew. Chem. Int. Edit 39, 1376-1398 (2000)). Materials comprising both the structural features of mesoporous silica and the properties of peptides constitute a novel class of bioorganic-inorganic hybrid materials which will find their place in techniques of catalysis, separation and molecular recognition, for example.

Hybrid materials can be obtained by a direct synthetic approach involving the functionalization of silica, i.e., by copolymerization of tetraethylorthosilicates (TEOS) and an organotrialkoxysilane having the desired function.

Alternatively, the organic fragment can be introduced by grafting it onto already-structured silica material (post-synthesis).

These two methods have been used to graft organic groups onto silica nanoparticles (Chandran, S. P., et al., Curr. Sci. India, 95, 1327-1333 (2008)), but also onto the inner surface of pores of ordered mesoporous silica (Wei et al., Materials 3, 4066-4079 (2010)). The synthesis can be carried out in the presence of surfactants, making it possible to control on a nanometer scale the structure and pore size of the materials obtained (Kresge, C. T. et al., Nature 359,710-712 (1992)).

The use of mesoporous silicas makes it possible to rationalize the inclusion of organic compounds, which can be more or less complex compounds, in these porous structures. This distinguishes hybrid mesoporous materials from those whose porosity is not controlled, such as silica gels. Thus, compounds of a certain complexity, such as enzymes or heme proteins, have been grafted in such structures (Zhao, X. S. et al., Materials Today 9, 32-39 (2006)). More recently, homopeptide nanocomposite materials were prepared by polymerization of various amino acid N-carboxyanhydrides (NCAs) on pores of mesoporous silica functionalized by amine functional groups obtained by grafting or by direct synthesis (Lunn, J. D. et al., Chem Mater 21, 3638-3648, (2009), and Subra, G. et al., J. Mater Chem 21, 6321-6326, (2011)). However, this technique does not make it possible to control the size of the peptides grafted onto the support and, above all, does not make it possible to control the sequences of the peptides.

Patent application US2003/0135024 discloses the direct grafting of peptide strands on glass beads. The objective of US2003/0135024 is to produce peptides on supports, wherein said supports are volatilized at the conclusion of peptide synthesis. However, the teaching of document US2003/0135024 relates to peptide synthesis techniques and not to obtaining hybrid materials. For example, the teaching of document US2003/0135024 relates only to “glass beads”-type supports.

In the document Parr et al., “Silicon-Matrizen für die Festphasen-Peptidsynthese”, Liebigs Ann Chem., 1974, 655-666, peptides are grafted onto glass beads. However, the study developed in this article is limited to the search for novel peptide synthesis supports and not to a true strategy for searching for novel hybrid materials that can be used as such.

US 2011/02888252 discloses chemical structures which enable better adhesion of the material formed. However, under this scenario, the applications are highly targeted.

WO 2008/031108 provides an example of a ligand linked to an antiviral peptide, DiprotinA (Ile-Pro-Ile). Nevertheless, no test is carried out on a potential material obtained with this product. The other products described do not involve peptides.

The scientific article by Sabrina S. Jedlicka et al. (J. Mater. Chem., 2007, 17, 5058-5067) discloses neuroactive “silane peptides” for producing thin films, which are then used to modulate the phenotype of embryonic carcinoma stem cell line P19.

The study by Cohn Przybylowski et al. (J. Mater. Chem., 2012, 22, 10672-10683) relates to the design of biological interfaces that stimulate cell differentiation.

The article by Iria M. Rio-Echevarria et al. (J. Am. Chem. Soc., 2011, 133, 8-11) relates to particular peptides containing alpha-aminoisobutyric acid (Aib) known to stabilize alpha-helix structures. Moreover, functionalized nanoparticles are disclosed.

WO 00/461238 relates to synthesis on a solid support wherein the solid support can be vaporized when cleavage occurs.

The scientific article by Junmin Huang et al. (Anal. Chem. 2005, 77, 3301-3308) discloses multi-proline/multi-valine chains grafted by their N-terminus onto silica via a spacer group which can be condensed Ahx (aminohexanoic acid). These functionalized silicas are used as stationary phases in chiral liquid chromatography.

The article by Carmen Coll et al. (Angew. Chem. Int. Ed. 2011, 50, 2138-2140) relates to mesoporous silica nanoparticles functionalized by peptide strands, said peptide strands, under the action of protease, releasing host molecules.

US 2011/0092672 discloses nanoparticles comprising a peptide-silica molecular fragment on the surface of the nanoparticles.

On the other hand, none of these documents reveal antimicrobial/antibiotic/antifungal materials of proven effectiveness, or tools for diversifying the hybrid materials obtained by grafting onto the C-terminus of the peptide chain or onto the side chain of the peptides.

Thus, the full potential of such hybrid materials has not heretofore been completely exploited.

However, the present invention allows controlled grafting of molecules of interest with a wide-ranging potential, in terms of grafting rate and in terms of the nature of the chemical bonds between the peptide chains and the silica support, by means of the use of perfectly defined hybrid unit blocks. The present invention also makes it possible to synthesize materials ab initio using perfectly defined hybrid unit blocks as precursors, thus allowing the production of novel materials with innovative features.

### SUMMARY OF THE INVENTION

The object of the present invention relates to a peptide conjugate of formula (I):

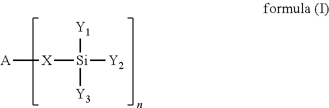

wherein:
A is a peptide fragment,
X is a spacer group preferably represented by a divalent radical derived from a saturated or unsaturated aliphatic hydrocarbon chain comprising from 1 to 10 carbon atoms, optionally intercalated with one or more structural linkers selected from arylene or fragments —O—, —S—, —C(═O)—, SO₂ or —N(R₁)—, wherein R₁ represents a hydrogen atom, an aliphatic hydrocarbon radical comprising from 1 to 6 carbon atoms, a benzyl radical or a phenethyl radical, wherein said chain is unsubstituted or is substituted by one or more radicals selected from halogen atoms, a hydroxyl group, alkyl radicals comprising from 1 to 4 carbon atoms or benzyl or phenethyl radicals,
Y₁, Y₂, Y₃, identical or different, each independently represents a hydrogen atom, a halogen atom, or an OR₂ radical wherein R₂ represents a hydrogen atom, an aryl group or a saturated or unsaturated aliphatic hydrocarbon chain comprising from 1 to 6 carbon atoms optionally substituted by an aryl, halogen or hydroxyl group,
   n is an integer between 1 and 50, preferably between 1 and 10. Preferably, the peptide conjugate (I) is not one of the following structures: H-YGGFLR-NH—CH₂—CH₂—CH₂—Si(OH)₃, H-YGGFLR-NH—CH₂—CH₂—CH₂—Si(OH)₂F or H-YGGFLR-NH—CH₂—CH₂—CH₂—Si(OH)F₂.

In particular, the object of the present invention relates to a peptide conjugate of formula (I):

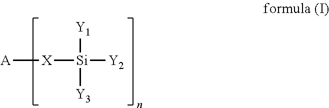

wherein:
A is a peptide fragment,
X is a spacer group preferably represented by a divalent radical derived from a saturated or unsaturated aliphatic hydrocarbon chain comprising from 1 to 10 carbon atoms, optionally intercalated with one or more structural linkers selected from arylene or fragments —O—, —S—, —C(═O)—, SO₂ or —N(R₁)—, wherein R₁ represents a hydrogen atom, an aliphatic hydrocarbon radical comprising from 1 to 6 carbon atoms, a benzyl radical or a phenethyl radical, wherein said chain is unsubstituted or is substituted by one or more radicals selected from halogen atoms, a hydroxyl group, alkyl radicals comprising from 1 to 4 carbon atoms or benzyl or phenethyl radicals,
Y₁, Y₂, Y₃, identical or different, each independently represents a hydrogen atom, a halogen atom, or an OR₂ radical wherein R₂ represents a hydrogen atom, an aryl group or a saturated or unsaturated aliphatic hydrocarbon chain comprising from 1 to 6 carbon atoms optionally substituted by an aryl, halogen or hydroxyl group,
n is an integer between 1 and 50, preferably between 1 and 10, characterized in that:
   if the peptide conjugate of formula (I), wherein A is a linear peptide fragment, comprises only one Si carried by an X group on the alpha amine at the N-terminus of fragment A then
      A is a peptide fragment selected from the group consisting of an antibiotic, an antimicrobial, an antifungal, an anti-inflammatory, a catalyst, a biological receptor ligand and an enzyme inhibitor, preferably an antibiotic, an antimicrobial, an antifungal, an anti-inflammatory and a catalyst, more preferably an antibiotic, an antimicrobial, an antifungal and an anti-inflammatory, even more preferably an antibiotic, an antimicrobial and an antifungal, even more preferably an antibiotic and an antimicrobial;
   or
      Y₁ is different from Y₂ and/or Y₃
   the peptide conjugate (I) is not one of the following structures: H—YGGFLR-NH—CH₂—CH₂—CH₂—Si(OH)₃, H-YGGFLR-NH—CH₂—CH₂—CH₂—Si(OH)₂F or H-YGGFLR-NH—CH₂—CH₂—CH₂—Si(OH)F₂,
      except for the following peptide conjugates: H-YGGFLR-NH—CH₂—CH₂—CH₂—Si(OH)₃, H-YGGFLR-NH—CH₂—CH₂—CH₂—Si(OH)₂F and H-YGGFLR-NH—CH₂—CH₂—CH₂—Si(OH)F₂.

More particularly, the object of the present invention relates to a peptide conjugate as described above characterized in that:

if the peptide conjugate of formula (I), wherein A is a linear peptide fragment, comprises the fragment of formula (If):

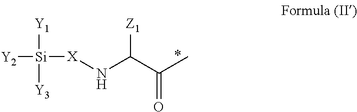

wherein,
Y₁, Y₂, Y₃ and X are as defined above,
Z₁ represents a side chain of a natural amino acid optionally substituted by a protective group,
* represents the bond whereby fragment (If) is linked to the rest of the peptide conjugate,
then A is a peptide fragment selected from the group consisting of an antibiotic, an antimicrobial, an antifungal, an anti-inflammatory, a catalyst, a biological receptor ligand and an enzyme inhibitor, preferably an antibiotic, an antimicrobial, an antifungal, an anti-inflammatory and a catalyst, more preferably an antibiotic, an antimicrobial, an antifungal and an anti inflammatory, even more preferably an antibiotic, an antimicrobial and an antifungal, even more preferably an antibiotic and an antimicrobial,
   preferably except for the structure H-YGGFLR-NH—CH₂—CH₂—CH₂—Si(OH)₃, H-YGGFLR-NH—CH₂—CH₂—CH₂—Si(OH)₂F and/or H-YGGFLR-NH—CH₂—CH₂—CH₂—Si(OH)F₂

Another object of the present invention relates to a method α of synthesizing peptide conjugates defined above (including the structures H-YGGFLR-NH—CH₂—CH₂—CH₂—Si(OH)₃, H-YGGFLR-NH—CH₂—CH₂—CH₂—Si(OH)₂F and/or H-YGGFLR-NH—CH₂—CH₂—CH₂—Si(OH)F₂) characterized in that it comprises the steps:
i) synthesis of a peptide strand A by standard peptide synthesis techniques, wherein said peptide strand A contains at least one reactive functional group not protected by a protective group,
ii) reaction in solution of peptide strand A containing at least one reactive functional group not protected by a protective group according to step i), with a reagent of formula (VI):

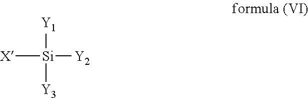

wherein
X′ is an X group as defined above activated for example by means of an isocyanate, an azide, an aldehyde, an activated carboxylic acid such as acyl chloride, or a —CO—NH—NH₂ group,
Y₁, Y₂, Y₃ are as defined above.

Thus, an aspect of the present invention relates to the use of a peptide conjugate defined above in order to incorporate a peptide strand of formula A in a silica material or a metal oxide.

The object of the present invention thus also relates to a synthetic mixture intended for the manufacture of peptide-silica hybrid materials characterized in that:
said synthetic mixture contains at least one peptide conjugate as defined herein;
said synthetic mixture optionally contains a preferably organic or inorganic solvent,
said synthetic mixture optionally contains another organic or inorganic monomer or polymer,
   said synthetic mixture optionally containing a catalyst that enables polymerization.

The object of the present invention also relates to a method β characterized by the following steps:
i) activation of any one of the Y₁, Y₂, and/or Y₃ groups of the peptide conjugate defined above, preferably by hydrolysis,
ii) condensation, optionally in situ, of the peptide conjugate obtained according to step i) on a support material, preferably selected from silica, mesoporous silica, silica nanoparticles, glass, metal oxide,
iii) optional rinsing step, preferably with water-miscible organic solvent such as DMF, acetone, DMSO,
iv) optional step of deprotecting the peptide strand, preferably with trifluoroacetic acid (TFA).

Thus, the object of the present invention relates to a grafted silica material β of peptide strands A as defined above which can be obtained by method 13 defined above, except for cases where:
peptide strand A is a peptide sequence consisting of a poly-Ala, poly-Lys, poly-Met, poly-Glu(OBzl) or poly-Glu fragment,
the support material consists of glass beads, or consists of glass, and/or
the spacer has the following structure:

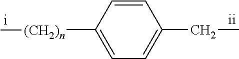

wherein n=0, 1, 2, etc.,
the “i” bond is attached to Si, and
the “ii” bond is attached to the peptide chain.

Preferably, the silica copolymer material β of peptide strands A does not comprise a protected (Boc strategy in particular) or deprotected YGGFLR fragment.

Preferably, the silica copolymer material β of peptide strands A does not comprise a poly-Pro fragment, in particular a proline dimer, a proline trimer and/or a proline tetramer.

Preferably, the silica copolymer material β of peptide strands A does not comprise a poly-Val fragment.

Preferably, the silica copolymer material β of peptide strands A does not comprise a monomer, dimer or trimer of the protected (Fmoc strategy in particular) or deprotected GDEVDG fragment.

Preferably, the silica copolymer material β of peptide strands A does not comprise the protected (Fmoc strategy in particular) or deprotected AAEAYAKELAEANMAKG fragment.

Preferably, the silica copolymer material β of peptide strands A does not comprise the protected (Fmoc strategy in particular) or deprotected Cys-Lys-Gly-Arg-Gly-Asp fragment.

Thus, the object of the present invention also relates to the use of the material β defined above to catalyze chemical reactions, to separate products by chromatography, to functionalize nanoparticles, to obtain biocompatible matrices for treating wounds and/or burns, to obtain material allowing facilitated electronic or ionic transport, to manufacture nanosensors, to manufacture printed circuits, to prepare antimicrobial surfaces, to prepare surfaces that promote cell regrowth in order to cover medical devices or silica particles used in the formulation of cosmetics.

An object of the present invention also relates to a method γ characterized by the following steps:
i) activation of any one of the Y₁, Y₂, and/or Y₃ groups of the peptide conjugate as defined above, preferably by hydrolysis,
ii) condensation, optionally in situ, of the peptide conjugate obtained according to step i) with a silica precursor, such as silicic acid, a silicate or a C₁-C₁₀ tetraalkoxysilane, more particularly tetraethoxysilane,
iii) optional rinsing step, preferably with water-miscible organic solvent such as DMF, acetone, DMSO,
iv) optional step of deprotecting the peptide strand, preferably with trifluoroacetic acid (TFA).

Thus, the object of the present invention relates to a silica copolymer material γ of peptide strands A as defined above which can be obtained by method γ, wherein is advantageously the silica copolymer material γ of peptide strands A is a mesoporous silica, a film, a gel, a suspension or a solution.

Preferably, the silica copolymer material γ of peptide strands A does not comprise a poly-Ala, poly-Lys, poly-Met, poly-Glu(OBzl) or poly-Glu fragment.

Preferably, the silica copolymer material γ of peptide strands A does not comprise a poly-Pro fragment, in particular a proline dimer, a proline trimer and/or a proline tetramer.

Preferably, the silica copolymer material γ of peptide strands A does not comprise a poly-Val fragment, in particular a valine dimer.

Preferably, the silica copolymer material γ of peptide strands A does not comprise a protected (Boc strategy in particular) or deprotected YGGFLR fragment.

Preferably, the silica copolymer material γ of peptide strands A does not comprise a GDEVDG fragment monomer, dimer or trimer.

Preferably, the silica copolymer material γ of peptide strands A does not comprise the protected (Fmoc strategy in particular) or deprotected AAEAYAKELAEANMAKG fragment.

Preferably, the silica copolymer material γ of peptide strands A does not comprise the protected (Fmoc strategy in particular) or deprotected Cys-Lys-Gly-Arg-Gly-Asp fragment.

Thus, the object of the present invention also relates to the use of the material γ as defined above to catalyze chemical reactions, to separate products by chromatography, to functionalize nanoparticles, to obtain biocompatible matrices for treating wounds and/or burns, to manufacture nanosensors, to manufacture printed circuits, to prepare antimicrobial surfaces, or to prepare surfaces that promote cell regrowth in order to cover medical devices.

Another object of the present invention relates to a methods characterized by the following steps:
i) activation of any one of the Y₁, Y₂, and/or Y₃ groups of the peptide conjugate as defined above, preferably by hydrolysis,
ii) self-condensation of the peptide conjugate obtained according to step i),
iii) optional rinsing step, preferably with water-miscible organic solvent such as DMF, acetone, DMSO,
iv) optional step of deprotecting the peptide strand, preferably with trifluoroacetic acid (TFA).

Thus, the object of the present invention relates to a silica copolymer material ε of peptide strands A as defined above which can be obtained by method ε.

Preferably, the silica copolymer materials of peptide strands A does not comprise a poly-Ala, poly-Lys, poly-Met, poly-Glu(OBzl) or poly-Glu fragment.

Preferably, the silica copolymer materials of peptide strands A does not comprise a poly-Pro fragment, in particular a proline dimer, a proline trimer and/or a proline tetramer.

Preferably, the silica copolymer materials of peptide strands A does not comprise a poly-Val fragment, in particular a valine dimer.

Preferably, the silica copolymer materials of peptide strands A does not comprise a protected (Boc strategy in particular) or deprotected YGGFLR fragment.

Preferably, the silica copolymer materials of peptide strands A does not comprise a GDEVDG fragment monomer, dimer or trimer.

Preferably, the silica copolymer materials of peptide strands A does not comprise the protected (Fmoc strategy in particular) or deprotected AAEAYAKELAEANMAKG fragment.

Preferably, the silica copolymer materials of peptide strands A does not comprise the protected (Fmoc strategy in particular) or deprotected Cys-Lys-Gly-Arg Gly-Asp fragment.

Thus, the object of the present invention also relates to the use of materials as defined above to catalyze chemical reactions, to separate products by chromatography, to functionalize nanoparticles, to obtain biocompatible matrices for treating wounds and/or burns, to obtain material allowing facilitated electronic or ionic transport, to prepare antimicrobial surfaces, to prepare surfaces that promote cell regrowth in order to cover medical devices or silica particles used in the formulation of cosmetics.

### DEFINITIONS

### Peptide Conjugate

The term “conjugate” refers to the fragment “A” linked to the rest of the molecule according to formula I.

The term “peptide” should be understood to mean a polymer of amino acids, to said amino acids being linked together by a peptide and/or pseudopeptide bond. A peptide generally contains between 2 and 80 to 100 amino acids, the upper limit not being clearly defined. Fragment A contains between 2 and 80 amino acids, more preferably between 3 and 40, and even more preferably between 4 and 20.

### Spacer

By “spacer” group is meant a fragment comprising at least one atom. Preferably, the spacer group contains at least one carbon atom. Advantageously the spacer group decreases steric hindrance between the peptide and the Si. More advantageously, the spacer group allows the silicate group to react with limited hindrance from fragment A. Moreover, the spacer group allows a stable bond between fragment A and Si, while allowing the silicate fragment to react. It is thus clear that the spacer group cannot be an amino acid residue (i.e., a condensed amino acid), or a peptide chain residue (i.e., a condensed peptide).

Advantageously, the spacer group comprises, or consists of, a saturated or unsaturated aliphatic hydrocarbon chain. The spacer group can also include heteroatoms, in particular selected from N, O, S or P, and can also be substituted, in particular by halogen atoms, or by hydroxyl, aryl, C₁-C₄ alkyl, sulfate, amine or phosphate groups. However, if heteroatoms are present in the spacer group, preferably said heteroatoms are not linked directly to Si.

Preferably, the spacer group is a linear or branched C₁-C₄ alkyl fragment. More preferably, the spacer group is a —(CH₂)₂—, —(CH₂)₃— or —(CH₂)₄— fragment. Even more preferably, the spacer group is a —(CH₂)₃— fragment.

### Saturated or Unsaturated Aliphatic Hydrocarbon Chain

By “saturated or unsaturated aliphatic hydrocarbon chain” is meant fragments of type C₁-C₁₀ alkyl, C₂-C₁₀ alkene or C₂-C₁₀ alkyne.

C₁-C₁₀ Alkyl

In the present invention, “C₁-C₁₀ alkyl” or “alkyl of 1 to 10 carbon atoms” refers to a linear or branched cyclic saturated aliphatic group comprising from 1 to 10 carbon atoms, such as for example a methyl, ethyl, isopropyl, tert-butyl, n-pentyl group, etc.

C₂-C₁₀ Alkene

By “C₂-C₁₀ alkene” or “alkene of 2 to 10 carbon atoms” is meant, in the context of the present invention, a linear or branched mono- or polyunsaturated aliphatic group comprising from 2 to 10 carbon atoms. An alkene group according to the invention comprises, preferably, one or more ethylenic unsaturations. For example, mention may be made of the groups ethylene, propylene, propyl-2-ene or propyl-3-ene, butylene, etc.

C₂-C₁₀ Alkyne

By “C₂-C₁₀ alkyne” or “alkyne of 2 to 10 carbon atoms” is meant, in the context is of the present invention, a linear or branched aliphatic group comprising from 2 to 10 carbon atoms and at least one double unsaturation, i.e., a triple bond between two carbon atoms. An alkyne group according to the invention comprises, preferably, one or more double unsaturations. For example, mention may be made of the groups acetylene, propyne, butyne, etc.

C₁-C₁₀ Alcohol
In the present invention, “C₁-C₁₀ alcohol” means a linear or branched saturated aliphatic C₁-C₁ alkyl group as defined above comprising at least one OH group. For example, alcohols can be ethanol, isopropanol, propanol, butanol, isobutanol, tertbutanol, etc.
C₃-C₁₀ Ketone
In the present invention, “C₃-C₁₀ ketone” means a linear, cyclic or branched saturated aliphatic C₁-C₁₀ alkyl group as defined above comprising at least one carbonyl group inserted between two carbons. For example, a ketone group can be acetone, butan-2-one or pentan-2-one.
C₄-C₁₀ diketone
In the present invention, “C₁-C₁₀ diketone” means two juxtaposed or non-juxtaposed carbonyl groups included in a C₂-C₈ alkyl chain. For example, a diacetone group can be acetylacetone.
C₃-C₁₀ Ester
In the present invention, “C₃-C₁₀ ester” refers to a C₁-C₈ alkyl group linked covalently to another C₁-C₈ alkyl group via a COO group, the total number of carbons being between 3 and 10. For example, an ester group can be ethyl acetate.
C₂-C₁₀ Ether
In the present invention, “C₁-C₁₀ ether” means a C₁-C₉ alkyl group linked covalently to another C₁-C₉ alkyl group via an oxygen, the total number of carbons being between 2 and 10. For example, an ether group can be diethyl ether.
C₁-C₁₀ Halogenoalkyl
In the present invention, “C₁-C₁₀ halogenoalkyl” means a C₁-C₁₀ alkyl group linked covalently to one or more halogen atoms, such as a chlorine, fluorine, iodine or bromine atom. For example, a halogenoalkyl group can be dichloromethane, chloroform or methyl iodide.
C₁-C₁₀ Alkyl Substituted by One or More Nitriles
In the present invention, “C₁-C₁₀ alkyl substituted by one or more nitriles” means a C₁-C₁₀ alkyl group as defined above linked covalently to one or more CN groups, such as acetonitrile.

### Cyclic Lactam

In the present invention, “cyclic lactam” means a C₃-C₁₀ alkyl ring in which is inserted a CO—NH group, optionally substituted by a C₁-C₁₀ alkyl group, such as, for example, a methyl, ethyl, isopropyl, tert-butyl, n-pentyl group, etc. An example of a cyclic lactam can be N-methyl-2-pyrrolidone (NMP).

### Arylene

An “arylene” group represents a substituent of an organic compound derived from an aryl fragment wherein at least one hydrogen atom has been removed from two carbons included in the aryl. Preferably, it is a phenethyl group.

### Aryl

By “aryl” group is meant an aromatic group, preferably consisting of from 5 to 10 carbon atoms, comprising one or more rings and optionally comprising one or more heteroatoms, in particular oxygen, nitrogen or sulfur, such as, for example, a phenyl, furan, indol, pyridine, naphthalene group, etc.

### Silica Material

By “silica material” is meant silica in various forms, such as common commercial silica, for example Corning®7980 silica, mesoporous silica, whether ordered or disordered, and SiO₂ nanoparticles.

Preferably the support is mesoporous silica, such as SBA, MCA, or HMS silica.

### Metal Oxide

By “metal oxide” is meant metal oxides in the general sense such as TiO₂, SnO₂, ZnO, Fe₂O₃ or mixtures thereof.

### Antimicrobial Activity

“Antimicrobial activity” according to the present invention is the generic definition as understood by the person skilled in the art, i.e., an effect relating to an antimicrobial agent. An antimicrobial (agent) is a substance that kills, slows the growth is of or blocks the growth of one or more microbes. By “growth” is meant in the context of the present invention any cellular operation allowing the cell to increase in volume, allowing the cell to divide or allowing the cell to reproduce. A microbe in the context of the present invention is any unicellular or multicellular organism pathogenic or parasitic to other living organisms such as humans.

Antibiotic Activity

“Antibiotic activity” according to the present invention is the generic definition as understood by the person skilled in the art, i.e., an effect relating to an antibiotic agent. An antibiotic (agent) is a substance that kills, slows the growth of or blocks the growth of one or more bacteria. By “growth” is meant in the context of the present invention any cellular operation allowing the cell (bacterium) to increase in volume, allowing the cell (bacterium) to divide or allowing the cell (bacterium) to reproduce.

### Antifungal Activity

“Antifungal activity” according to the present invention is the generic definition as understood by the person skilled in the art, i.e., an effect relating to an antifungal agent. An antifungal (agent) is a substance that kills, slows the growth of or blocks the growth of at least one fungus. By “growth” is meant in the context of the present invention any cellular operation allowing the cell (fungus) to increase in volume, allowing the cell (fungus) to divide or allowing the cell (fungus) to reproduce.

### Antimicrobial Surfaces

By “antimicrobial surfaces” is meant in the present invention that the surfaces described in the present manuscript have an anti-adhesive, anti-biofilm effect simultaneously with or not simultaneously with a cell lysis, slowed cell growth and/or blocked cell growth effect in microbes.

### Antibiotic Surfaces

By “antibiotic surfaces” is meant in the present invention that the surfaces described in the present manuscript have an anti-adhesive, anti-biofilm effect to simultaneously with or not simultaneously with a bactericidal, bacteriostatic effect (the bacteria are lysed and can no longer divide or grow and/or reproduce).

### Antifungal Surfaces

By “antifungal surfaces” is meant in the present invention that the surfaces is described in the present manuscript have an anti-adhesive, anti-biofilm effect simultaneously with or not simultaneously with a cell lysis, slowed cell growth and/or blocked cell growth effect in fungi.

### Natural Peptide

A natural peptide is a peptide found in the environment without direct human intervention (except its extraction/isolation).

### Synthetic Peptide

A synthetic peptide is a peptide not found in the environment without direct human intervention (except its extraction/isolation). For example, a synthetic peptide can be a sequence of a natural peptide wherein at least one natural amino acid has been replaced by another natural or synthetic amino acid.

### Linear/Cyclic Peptide

By linear peptide is meant that all the amino acids of the peptide are linked in their sequential order and that the peptide has an N-terminus and a C-terminus.

Generally, by cyclic peptide is meant an amino acid sequence without an N-terminus or C-terminus. In the context of the present invention, a cyclic peptide can be linked by a side chain to the solid support, and so the general definition applies, or it can be linked to the support by one of its N-terminal or C-terminal ends and then the ring is closed by means of at least one amino acid side chain.

Peptides linked by an S—S(or Se—Se) bridge, some of which are called cyclic, are not included in the present definition of cyclic peptide. Peptides having an N-terminus and a C-terminus, an S—S or Se—Se bridge, are not regarded in the present invention as cyclic peptides. They are included in the category either of synthetic peptides or of natural peptides.

### Pseudopeptide

A pseudopeptide is a peptide comprising at least one pseudopeptide bond. A pseudopeptide bond links two amino acids in a way different from the (C═O)—(NH) bond. One of the two amino acids can thus be non-natural or replaced by a non-amino acid analog having functional groups required for the pseudopeptide bond such as, for example, a diamine or a malonate type diacid. In the context of the present invention, pseudopeptide bonds are advantageously selected from (NH)—(C═O)—(C═O)—(NH—NH)—, —(C═O)—(N(OH))—, —(C═O)—(N(C₁-C₆ alkyl))-, in particular is —(C═O)—(N(CH₃))—, —(C═O)—(N(C₁-C₆ alkyl substituted by OH))—, —(C═O)—(N(NH₂))—, —(C═O)—(CH₂)—, —(C═O)—O—, —(C═O)—S—, —(C═S)—(NH)—, —(C═S)—(NH—NH)—, —(C═S)—(N(OH))—, —(C═S)—(N(C₁-C₆ alkyl))-, in particular —(C═S)—(N(CH₃))—, —(C═S)—(N(C₁-C₆ alkyl substituted by a OH))—, —(C═S)—(N(NH₂))—, —(C═S)—(CH₂)—, —(C═S)—O—, —(C═S)—S—, —(C≡CH₂)—(NH)—, —(C≡CH₂)—(NH—NH)—, —(C≡CH₂)—(N(OH))—, —(C≡CH₂)—(N(C₁-C₆ alkyl))-, in particular —(C≡CH₂)—(N(CH₃))—, —(C≡CH₂)—(N(C₁-C₆ alkyl substituted by OH))—, —(C≡CH₂)—(N(NH₂))—, —(C≡CH₂)—(CH₂)—, —(C≡CH₂)—O—, —(C≡CH₂)—S—, —(C≡NH)—(NH)—, —(C≡NH)—(NH—NH)—, —(C≡NH)—(N(OH))—, —(C≡NH)—(N(C₁-C₆ alkyl))-, in particular —(C≡NH)—(N(CH₃))—, —(C≡NH)—(N(C₁-C₆ alkyl substituted by OH))—, —(C≡NH)—(N(NH₂))—, —(C≡NH)—(CH₂)—, —(C≡NH)—O—, —(C≡NH)—S—, —(CH₂)—(NH)—, —(CH₂)—(NH—NH)—, —(CH₂)—(N(OH))—, —(CH₂)—(N(C₁-C₆ alkyl))-, in particular —(CH₂)—(N(CH₃))—, —(CH₂)—(N(C₁-C₆ alkyl substituted by OH))—, —(CH₂)—(N(NH₂))—, —(CH₂)—(CH₂)—, —(CH₂)—O—, —(CH₂)—S—, —(CH(OH))—(NH)—, —(CH(OH))—(NH—NH)—, —(CH(OH))—(N(OH))—, —(CH(OH))—(N(C₁-C₆ alkyl))-, in particular —(CH(OH))—(N(CH₃))—, —(CH(OH))—(N(C₁-C₆ alkyl substituted by OH))—, —(CH(OH))—(N(NH₂))—, —(CH(OH))—(CH₂)—, —(CH(OH))—O—, —(CH(OH))—S—, —(CH)═(CH)—, —(CH)═N—NH—, —(CH)═N—.

The preferred pseudopeptide bonds according to the present invention are —(C═O)—(N(C₁-C₆ alkyl))-, in particular —(C═O)—(N(CH₃))—, —(C═O)—(NH—NH)—, —(C═O)—O—, —(CH₂)—(NH)—, —(C≡CH₂)—(NH)—, —(C═O)—O—, —(C═O)—S—, —(C═S)—(NH)—, —(CH)═(CH)—, —(C═O)—(N(OH))—.

When pseudopeptide bonds are formed, the various reactive groups of the amino acids can also be protected. The term “pseudopeptides” thus also comprises compounds having pseudopeptide bonds whose reactive groups, such as those of the side chains, are protected.

These protective groups are groups known to the person skilled in the art. These protective groups and use thereof are described in work such as, for example, Greene, “Protective Groups in Organic Synthesis”, Wiley, New York, 2007 4^{th} edition; Harrison et al., “Compendium of Synthetic Organic Methods”, Vol. 1 to 8 (J. Wiley & Sons, 1971 to 1996). Moreover, peptide synthesis techniques are described in Paul Lloyd to Williams, Fernando Albericio, Ernest Giralt, “Chemical Approaches to the Synthesis of Peptides and Proteins”, CRC Press, 1997 or Houben-Weyl, “Methods of Organic Chemistry, Synthesis of Peptides and Peptidomimetics”, Vol. E 22a, Vol. E 22b, Vol. E 22c, Vol. E 22d., M. Goodmann Ed., Georg Thieme Verlag, 2002.

An example of a preferred pseudopeptide is depsipeptides, i.e., peptides in which is at least one peptide bond has been replaced by an ester bond —COO—.

### Amino Acid

The expression “amino acid” refers to any molecule having at least one carboxylic acid, at least one amine and at least one carbon linking said amine and said carboxylic acid. Preferably, the amino acids which can be used in the context of the present invention are so-called “natural” amino acids and/or synthetic amino acids as defined below. Preferably, the amino acids of the present invention are L-amino acids.

### Natural Amino Acid

The expression “natural” amino acid represents, among other things, the following amino acids: glycine (Gly), alanine (Ala), valine (Val), leucine (Leu), isoleucine (Ile), serine (Ser), threonine (Thr), phenylalanine (Phe), tyrosine (Tyr), tryptophan (Trp), cysteine (Cys), methionine (Met), proline (Pro), aspartic acid (Asp), asparagine (Asn), glutamine (Gln), glutamic acid (Glu), histidine (His), arginine (Arg) and lysine (Lys). The preferred natural amino acids according to the present invention are L-amino acids.

### Synthetic Amino Acid

By synthetic amino acid is meant all non-natural amino acids as defined above. These synthetic amino acids can be selected from: β-alanine, allylglycine, tert-leucine, norleucine (Nle), 3-aminoadipic acid, 2-aminobenzoic acid, 3-aminobenzoic acid, 4-acid, 2-aminobutanoic acid, 4-aminolcarboxymethyl piperidin, 1-amino-1-cyclobutanecarboxylic acid, 4-aminocyclohexaneacetic acid, 1-amino-1-cyclohexanecarboxylic acid, (1R,2R)-2-aminocyclohexanecarboxylic acid, (1R,2S)-2-aminocyclohexanecarboxylic acid, (1S,2R)-2-aminocyclohexanecarboxylic acid, (1S,2S)-2-aminocyclohexanecarboxylic acid, 3-aminocyclohexanecarboxylic acid, 4-aminocyclohexanecarboxylic acid, (1R,2R)-2-aminocyclopentanecarboxylic acid, (1R,2S)-2-aminocyclopentanecarboxylic acid, 1-amino-1-cyclopentanecarboxylic acid, 1-amino-1-cyclopropanecarboxylic acid, 3-aminomethylbenzoic acid, 4-aminomethylbenzoic acid, 2-aminobutanoic acid, 4-aminobutanoic acid, 6-aminohexanoic acid, 1-aminoindane-1-carboxylic acid, 2-aminoisobutyric acid (Aib), 4-aminomethyl-phenylacetic acid, 4-aminophenylacetic acid, 3-amino-2-naphthoic acid, 4-aminophenylbutanoic acid, 4-amino-5-(3-indolyl)-pentanoic acid, (4R,5S)-4-amino-5-methylheptanoic acid, (R)-4-amino-5-methylhexanoic acid, (R)-4-amino-6-methylthiohexanoic acid, (S)-4-amino-pentanoic acid, (R)-4-amino-5-phenylpentanoic acid, 4-aminophenylpropionic acid, (R)-4-aminopimeric acid, (4R,5R)-4-amino-5-hyroxyhexanoic acid, (R)-4-amino-5-hydroxypentanoic acid, (R)-4-amino-5-(p-hydroxyphenyl)-pentanoic acid, 8-aminooctanoic acid, (2S,4R)-4-amino-pyrrolidine-2-carboxylic acid, (2S,4S)-4-amino-pyrrolidine-2-carboxylic acid, azetidine-2-carboxylic acid, (2S,4R)-4-benzyl-pyrrolidine-2-carboxylic acid, (S)-4,8-diaminooctanoic acid, tert-butylglycine, γ-carboxyglutamate, β-cyclohexylalanine, citrulline, 2,3-diamino propionic acid, hippuric acid, homocyclohexylalanine, moleucine, homophenylalanine, 4-hydroxyproline, indoline-2-carboxylic acid, isonipecotic acid, α-methyl-alanine, naphthyl-alanine, nicopetic acid, norvaline, octahydroindole-2-carboxylic acid, ornithine (Orn), penicillamine, phenylglycine (Phg), 4-phenyl-pyrrolidine-2-carboxylic acid, propargylglycine, 3-pyridinylalanine, 4-pyridinylalanine, 1-pyrrolidine-3-carboxylic acid, sarcosine, statins, tetrahydroisoquinoline-1-carboxylic acid, 1,2,3,4-tetrahydroisoquinoline-3-carboxylic acid, tranexamic acid, 4,4-difluoro proline, 4-fluoro proline, alpha-(3,4-difluorobenzyl)-proline, gamma-(3,4-difluorobenzyl)-proline, alpha-(trifluoromethyl)phenylalanine, hexafluoroleucine, 5,5,5-trifluoroleucine, 6,6,6-trifluoronorleucine, 2-(trifluoromethyl)leucine, 2-(trifluoromethyl)norleucine, 4,4,4-trifluorovaline, 4,4,4,4′,4′,4′-hexafluorovaline, pentafluorophenylalanine, 2,3-difluorophenyl alanine, 2,4-difluorophenylalanine, 2,5-difluorophenylalanine, 2,6-difluorophenyl alanine, 3,4-difluorophenylalanine, 3,5-difluorophenylalanine, 3,3-difluoro-3-(4-fluorophenyl)alanine, 2,3-difluorophenylglycine, 2,4-difluorophenylglycine, 2,5-difluorophenylglycine, 3,4-difluorophenylglycine, 4,4-difluoroethylglycine, 4,4,4-trifluoroethylglycine, 4-fluorotryptophan, 5-fluorotryptophan, 6-fluorotryptophan, 5-methyltryptophan, S-tritylcysteine, selenocysteine, selenomethionine, ethionine, β-(2-thienyl)alanine, β-chloroalanine, thiazolylalanine, triazolalanine, p-fluorophenylalanine, o-fluorophenylalanine, m-fluorophenylalanine, dihydroxyphenylalanine, 2,5-dihydrophenylalanine, thioproline, pipecolic acid, canavanine, indospicine, 3,4-dehydroproline, histidinol and hexafluoronorleucine, and the like.

### Side Chain of an Amino Acid

The term “side chain of an amino acid” refers to the fragment carried by the a carbon of an amino acid. For example, the side chains of natural amino acids such as glycine, valine, alanine and aspartic acid correspond to the hydrogen atom and the groups isopropyl, methyl and CH₂—COOH, respectively.

The side chains of other amino acids can be included in the definition of side chain of an amino acid, such as those of the following amino acids: 4-amino tetrahydropyran-4-carboxylic acid, allylglycine, diamino butyric acid, diamino propionic acid, aminoserine, aminobutyric acid, amino butylglycine, phenylglycine, 4-fluorophenylalanine, 4-nitrophenylalanine, citrulline, cyclohexylalanine, thienylalanine, and the like.

Amino acid side chains can be protected by protective groups (P) and more particularly N-protective, O-protective or S-protective groups when these chains contain the corresponding heteroatoms. Certain reactive functional groups of peptides must be protected during the synthesis of said peptides. Indeed, peptides are typically synthesized via activation of the carboxylic acid functional group of an amino acid, or of a chain of amino acids, by means of a coupling agent. This activated acid is brought together with an amino acid, or a chain of amino acids, whose terminal amine is not protected, thus resulting in the formation of an amide bond, also called a peptide bond. The coupling conditions and the coupling agents used are very well-known to the person skilled in the art.

Protective groups (P) are also groups known to the person skilled in the art. These protective groups and use thereof are described in work such as, for example, Greene, “Protective Groups in Organic Synthesis”, Wiley, New York, 2007 4^{th} edition; Harrison et al., “Compendium of Synthetic Organic Methods”, Vol. 1 to 8 (J. Wiley & Sons, 1971 to 1996). Moreover, peptide synthesis techniques are described in Paul Lloyd-Williams, Fernando Albericio, Ernest Giralt, “Chemical Approaches to the Synthesis of Peptides and Proteins”, CRC Press, 1997 or Houben-Weyl, “Methods of Organic Chemistry, Synthesis of Peptides and Peptidomimetics”, Vol. E 22a, Vol. E 22b, Vol. E 22c, Vol. E 22d., M. Goodmann Ed., Georg Thieme Verlag, 2002. Protective groups carried by a nitrogen atom will be referred to as N-protective groups.

The same applies to S-protective and O-protective groups, etc. For example, hydroxyl can be protected by a trityl group, or carboxylic acid can be protected in the form of a tert-butyl ester. In the case of synthesis on a solid support, it is the resin which serves as a protective group to the C-terminal carboxylic functional group.

Protection of the amino group (i.e., “alpha amine”) of the amino acid can be carried out, for example, by a tert-butyloxycarbonyl group (hereinafter referred to as Boc-) or a -9-fluorenylmethyloxycarbonyl group (hereinafter referred to as Fmoc-) represented by the formula:

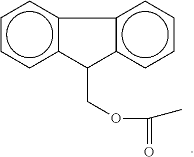

Protection is carried out according to known methods of the prior art. For m example, protection by the Boc-group can be obtained by reacting the amino acid with di-tert-butylpyrocarbonate (Boc₂O). When protecting functional groups of natural amino acids, the amino acids obtained are synthetic until the protective group(s) are removed, thus releasing the so-called natural amino acid.

### Standard Peptide Synthesis Techniques

Peptides are typically synthesized via activation of the carboxylic acid functional group of an amino acid, or a chain of amino acids, by means of a coupling agent. This activated acid is brought together with an amino acid, or a chain of amino acids, whose terminal amine is unprotected, thus resulting in the formation of an amide bond, also called a peptide bond. The coupling conditions and coupling agents used are well-known to the person skilled in the art and are described, for example, in work such as Greene, “Protective Groups in Organic Synthesis”, Wiley, New York, 2007 4^{th} edition; Harrison et al., “Compendium of Synthetic Organic Methods”, Vol. 1 to 8 (J. Wiley & Sons, 1971 to 1996). Moreover, peptide synthesis techniques are described in Paul Lloyd-Williams, Fernando Albericio, Ernest Giralt, “Chemical Approaches to the Synthesis of Peptides and Proteins”, CRC Press, 1997 or Houben-Weyl, “Methods of Organic Chemistry, Synthesis of Peptides and Peptidomimetics”, Vol. E 22a, Vol. E 22b, Vol. E 22c, Vol. E 22d., M. Goodmann Ed., Georg Thieme Verlag, 2002.

### DETAILED DESCRIPTION

In an embodiment of the present invention, the peptide conjugate of formula (I) is characterized in that peptide fragment A is a linear natural peptide strand, a linear synthetic peptide strand, a linear protected natural peptide strand, a linear protected synthetic peptide strand, a linear natural pseudopeptide strand, a linear synthetic pseudopeptide strand, a linear protected natural pseudopeptide strand, or a linear protected synthetic pseudopeptide strand, or peptide fragment A comprises or consists of a cyclic natural peptide fragment, a cyclic synthetic peptide fragment, a cyclic protected natural peptide fragment, a cyclic protected synthetic peptide fragment, a cyclic natural pseudopeptide fragment, a cyclic synthetic pseudopeptide fragment, a cyclic protected natural pseudopeptide fragment or a cyclic protected synthetic pseudopeptide fragment.

In an embodiment of the present invention, the peptide conjugate of formula (I) is characterized in that Y₁ represents a fragment different from Y₂, and/or Y₃. Advantageously, Y₁ represents an OR₂ radical wherein R₂ represents a hydrogen atom, an aryl group or a saturated or unsaturated aliphatic hydrocarbon chain comprising from 1 to 6 carbon atoms optionally substituted by an aryl, halogen or hydroxyl group and the Y₂ and/or Y₃ groups represent a hydrogen atom or a halogen atom.

More advantageously, Y₁ represents an OR₂ radical wherein R₂ represents methyl or ethyl, and the Y₂ and/or Y₃ groups represent a hydrogen atom or a halogen atom.

Still more advantageously, Y₁ represents an OR₂ radical wherein R₂ represents methyl or ethyl, and the Y₂ and/or Y₃ groups represent a hydrogen, fluorine or chlorine atom.

Most advantageously, Y₁ represents an OR₂ radical wherein R₂ represents methyl or ethyl, and the Y₂ and/or Y₃ groups represent a fluorine or chlorine atom.

In an embodiment of the present invention, the peptide conjugate of formula (I) is characterized in that Y₁ and Y₂ independently represent an OR₂ radical wherein R₂ represents a hydrogen atom, an aryl group or a saturated or unsaturated aliphatic hydrocarbon chain comprising from 1 to 6 carbon atoms optionally substituted by an aryl, halogen or hydroxyl group and the Y₃ groups represent a hydrogen atom or a halogen atom.

More advantageously, Y₁ and Y₂ independently represent an OR₂ radical wherein R₂ represents methyl or ethyl, and the Y₃ group represents a hydrogen atom or a halogen atom.

Still more advantageously, Y₁ and Y₂ independently represent an OR₂ radical wherein R₂ represents methyl or ethyl, and the Y₃ group represents a hydrogen, fluorine or chlorine atom.

Most advantageously, Y₁ and Y₂ independently represent an OR₂ radical wherein R₂ represents methyl or ethyl, and the Y₃ group represents a fluorine or chlorine atom.

In an embodiment of the present invention, the peptide conjugate of formula (I) is characterized in that peptide fragment A comprises between 2 and 80 amino acids, preferably between 2 and 30 amino acids.

In an embodiment of the present invention, the peptide conjugate of formula (I) is characterized in that the peptide fragment is an antibiotic, an antimicrobial, an antifungal, an antiviral, an anti-inflammatory, a catalyst, a structured peptide fragment, a biological receptor ligand and/or an enzyme inhibitor.

Advantageously, in an embodiment of the present invention, the peptide conjugate of formula (I) is characterized in that the peptide fragment is an antibiotic, an antimicrobial, an antifungal, an anti-inflammatory, a catalyst, a biological receptor ligand and/or an enzyme inhibitor.

More advantageously, in an embodiment of the present invention, the peptide conjugate of formula (I) is characterized in that the peptide fragment is an antibiotic, an antimicrobial, an antifungal and/or an anti-inflammatory.

Still more advantageously, in an embodiment of the present invention, the peptide conjugate of formula (I) is characterized in that the peptide fragment is an antibiotic, an antimicrobial and/or antifungal.

More advantageously still, in an embodiment of the present invention, the peptide conjugate of formula (I) is characterized in that the peptide fragment is an antibiotic and/or an antimicrobial.

In an embodiment of the present invention, the peptide conjugate of formula (I) is characterized in that the Si:N ratio in moles comprised in the conjugate is between 1:0.3 and 1:100, preferably between 1:1 and 1:30, and even more preferably between 1:2 and 1:15.

In an embodiment of the present invention, the peptide conjugate of formula (I) is characterized in that it comprises at least one of the fragments of the following formulas (II), (III) and/or (IV):

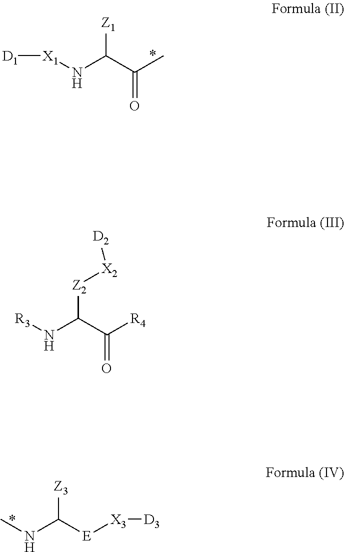

wherein,
D₁, D₂, D₃, identical or different, each independently represents a fragment of formula (V):

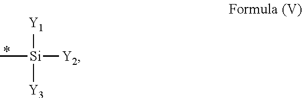

wherein, Y₁, Y₂, and Y₃ are as defined above,
X₁, X₂, X₃, identical or different, each independently represents a spacer group as defined above,
Z₁, Z₃, identical or different, each independently represents a side chain of a natural amino acid optionally substituted by a protective group,
Z₂ represents a side chain of a natural amino acid substituted by X₂ or a bond,
R₃ represents the N-terminal fragment of the peptide strand, a hydrogen atom or an N-protective group,
R₄ represents the C-terminal fragment of the peptide strand, a hydrogen atom, an NH₂ group, an —OR₅ group, wherein R₅ represents a hydrogen atom or an alkyl radical of 1 to 10 carbon atoms, or a carbonyl-activating atom or group such as a halogen atom or a succinimide group,
E represents the group (C═O)— or —NH—,
* represents the at least one bond whereby the fragments are linked to the rest of the peptide conjugate.

In a preferred embodiment, the groups Y₁, Y₂, and Y₃ represent OR₂ wherein R₂ represents a hydrogen atom, an aryl group or a saturated aliphatic hydrocarbon chain comprising from 1 to 6 carbon atoms. More preferably, R₂ represents a saturated aliphatic hydrocarbon chain comprising from 1 to 6 carbon atoms, even more preferably selected from methyl, ethyl and propyl.

In a preferred embodiment, silica material β is grafted of peptide strands A as defined above and can be obtained by method β, except for the case where the X group is any one of the following groups:

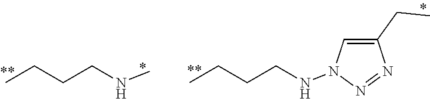

wherein:

* bonds represent bonds linked to the peptide fragment and

** bonds represent bonds linked to Si.

A particular embodiment of the present invention thus also relates to a synthetic mixture intended for the manufacture of antibiotic, antimicrobial, antifungal and/or anti-inflammatory peptide-silica hybrid materials characterized in that:
said synthetic mixture contains at least one peptide conjugate as defined herein;
said synthetic mixture optionally contains a solvent, preferably an organic solvent such as C₁-C₁₀ alcohol, C₃-C₁₀ ketone, C₄-C₁₀ diketone, C₃-C₁₀ ester, C₂-C₁₀ ether, C₁-C₁₀ halogenoalkyl, C₁-C₁₀ alkyl substituted by one or more nitriles, cyclic lactam optionally substituted by C₁-C₁₀ alkyl, aryl substituted by C₁-C₁₀ alkyl, formamide substituted by two C₁-C₁₀ alkyls, or an inorganic solvent such as water;
said synthetic mixture optionally contains another organic or inorganic polymer selected from SiZₚA₄₋ₚ and Z_{q}A_{3-q}Si—R_{B} and Z_{q}A_{3-q}Si—R_{B}-SiZ_{q}A_{3-q} wherein:
   Z and A are independently selected from a hydrogen, chlorine or bromine atom or a hydroxy, methoxy, ethoxy, phenoxy, methyl, ethyl, propyl or isopropyl group;
   p is 0, 1, 2 or 3;
   is 0, 1 or 2; and
   R_{B} represents a spacer preferably comprising a polyethylene glycol or poloxamer fragment (i. e., poly(ethylene glycol)-poly(propylene glycol)-poly(ethylene glycol) triblock copolymers) of mass between 400 to 50,000 daltons, preferably between 1,000 to 20,000 daltons, more preferably between 4,000 and 15,000 daltons, (for example, a P123® or F127® type triblock copolymer, which are poly(ethylene glycol)-poly(propylene glycol)-poly(ethylene glycol) copolymers of about 5,800 daltons and 12,600 daltons, respectively).
said synthetic mixture optionally contains a catalyst that allows polymerization, if need be, preferably selected from an inorganic acid, an organic acid, an inorganic base or an organic base, a metallic or organometallic complex.

Advantageously, the synthetic mixture intended for the manufacture of antibiotic, antimicrobial and/or antifungal peptide-silica hybrid materials as above is characterized in that:
said synthetic mixture optionally contains a solvent, preferably selected from acetone, acetylacetone, ethyl acetate, THF, Et₂O, iPr₂O, CHCl₃, CH₂—Cl₂, MeCN, NMP, DMSO, toluene and DMF, R_{A}OH wherein R_{A} can represent a hydrogen atom, a methyl, ethyl, propyl, isopropyl or butyl group.

Advantageously, the synthetic mixture intended for the manufacture of antibiotic, antimicrobial and/or antifungal peptide-silica hybrid materials as above is characterized in that said synthetic mixture contains an acid catalyst that allows polymerization, selected from HF, HCl, HBr, HI, HNO₃, H₂SO₄, CH₃COOH.

Advantageously, the synthetic mixture intended for the manufacture of antibiotic, antimicrobial and/or antifungal peptide-silica hybrid materials as above is characterized in that said synthetic mixture optionally contains a base catalyst that allows polymerization, selected from LiOH, NaOH, KOH, M₂CO₃ (M representing Li, Na, K, Cs and 0.5Mg)

Advantageously, the synthetic mixture intended for the manufacture of antibiotic, antimicrobial and/or antifungal peptide-silica hybrid materials as above is characterized in that said synthetic mixture optionally contains an MF type catalyst that allows polymerization wherein M represents Li, Na, K, Cs and a quaternary amine substituted by one or more groups, identical or different, selected from Me, Et, Pr, ⁱPr, Bu and ^{t}Bu.

### FIGURES

FIG. 1: LC spectrum of the hybrid peptide of example 1

FIG. 2: LC mass spectrum of the peak of FIG. 1 obtained at 0.76

FIG. 3: LC mass spectrum of the peak of FIG. 1 obtained at 1.29

### EXAMPLES

The examples below in no way limit the scope of the protection sought and are provided for the purpose of illustration of the present invention.

### Example 1

### Synthesis of Peptide-Silane Hybrid Unit Blocks: Synthesis of (EtO)₃Si(CH₂)₃NH—CO-Gly-Phe-Glu-NH₂

The peptide is synthesized on a solid support using a CEM Liberty™ type peptide synthesizer using 2,450 MHz microwave irradiation for the coupling and deprotection steps in a Fmoc/tert-butyl strategy.

The synthesis was carried out on a 0.25 mmol scale on Fmoc-Rink-amide polystyrene resin (390 mg, 0.640 mmol/g) on a 0.25 mmol scale. The coupling reactions are carried out with an excess of 5 equivalents of amino acid (0.2 M stock solution in DMF), 5 eq of HBTU (0.5 M stock solution in DMF), and 10 eq of DIEA (2 M stock solution in NMP solution).

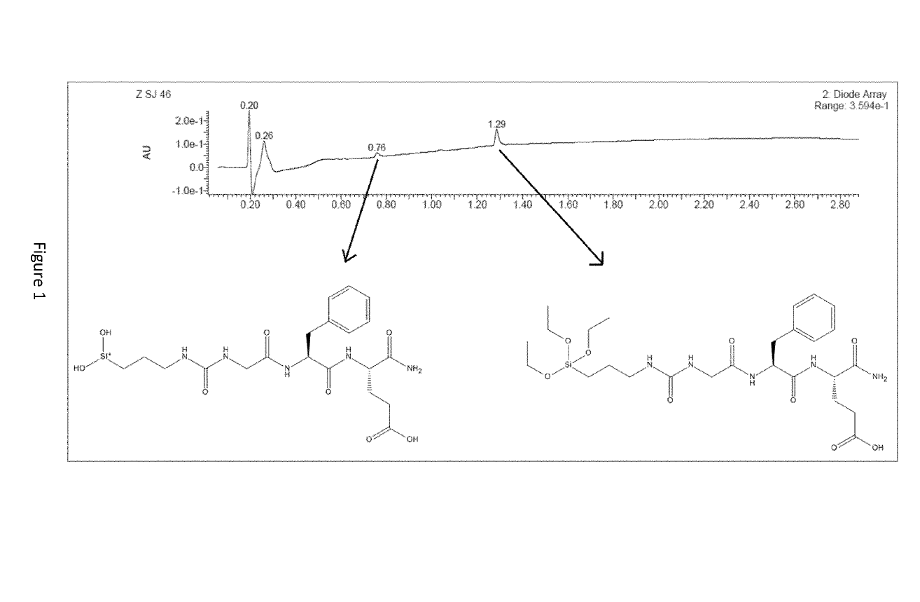

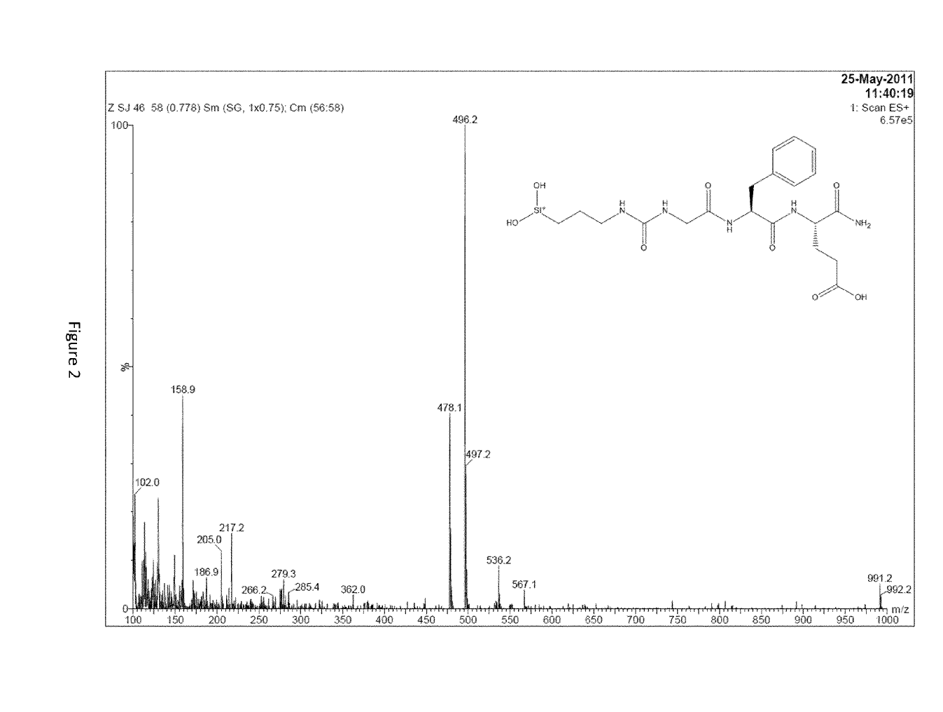

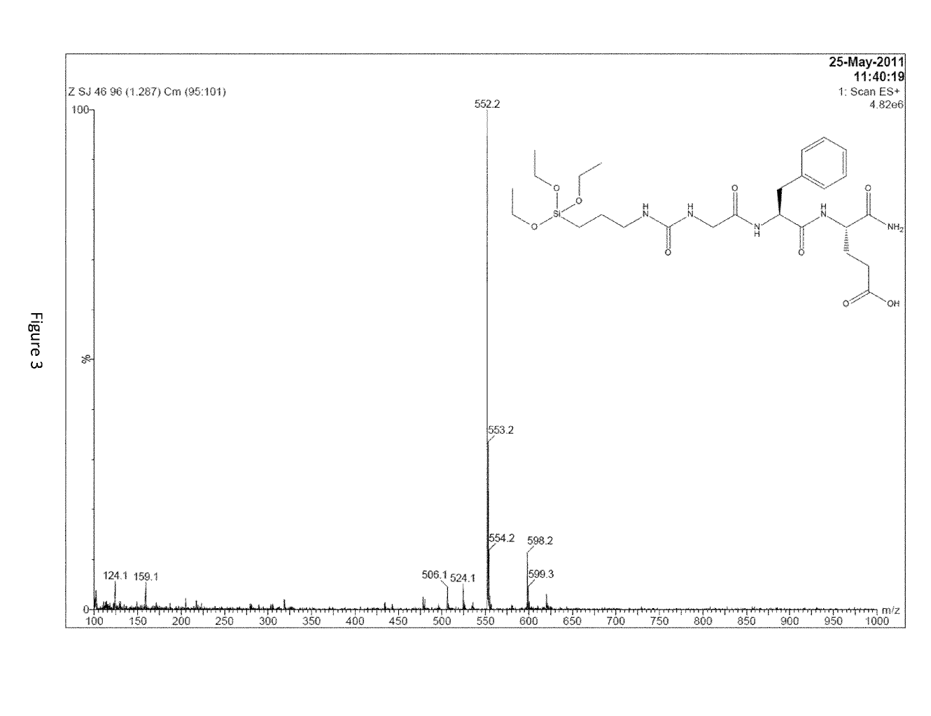

The cleavage of the resin is carried out for 90 minutes with stirring in trifluoroacetic acid. After evaporation of the solvent and trituration with ether, the peptide in the form of TFA salts (TFA, H-Gly-Phe-Glu-NH₂) is solubilized in 20 ml of a water/acetonitrile mixture, frozen then lyophilized (>95% purity determined by HPLC). 46.4 mg of TFA salts of the peptide (0.1 mmol) are then reacted in DMF solution containing DIPEA (10 eq) and 1.2 equivalents of 3-isocyanatopropyltriethoxy-silane (ICPTS) (29.7 mg) for 2 hours. Diethyl ether (about 100 ml) is added to precipitate the hybrid peptide (EtO)₃Si(CH₂)₃NH—CO-Gly-Phe-Glu-NH₂ (41.8 mg, 65% yield). The hybrid peptide was then characterized by LC/MS (cf. FIGS. 1, 2 and 3).

### Example 2

### Condensation of Hybrid Unit Blocks on Mesoporous Silica

This functionalization pathway consists in grafting hybrid unit blocks in the pores of mesostructured silicas by reacting these blocks with silanol groups present at the surface of the pores.

In a 50 ml round-bottom flask equipped with magnetic stirring, a known mass of mesostructured silica and a desired quantity of hybrid unit blocks in dimethylformamide are mixed. The suspension is left stirring for 1 hour at room temperature then 24 hours at 80° C. The solid is filtered then washed several times with dimethylformamide, with dichloromethane, with ethanol, then dried under vacuum.

### Example 3

### Condensation of Hybrid Unit Blocks Via Copolymerization

This functionalization pathway in the pores of mesostructured silica consists in the co-hydrolysis-polycondensation of hybrid unit blocks and tetraalkoxysilane in the presence of surfactant.

In a 250 ml Erlenmeyer flask, 4.0 g (0.69 mmol) of surfactant of block copolymer type of formula H—(O—CH₂—CH₂)₂₀(O—CH(CH₃)—CH₂—)₇₀(O—CH₂—CH₂)₂₀—OH (commonly called Pluronic P123) is dissolved in 160 ml of hydrochloric acid aqueous solution, pH 1.5. This solution is then added to a desired quantity of peptide-silane hybrid unit blocks (peptide-Si(OEt)₃) and tetraethoxysilane (TEOS). The mixture is left under vigorous and steady stirring at room temperature for 2 hours to allow a transparent solution, containing hydrolyzed precursors interacting with the surfactant, to be obtained. The reaction medium is then heated at 60° C. with stirring and immediately 80 mg of NaF catalyst is added. After a few minutes, the solution becomes cloudy and a white precipitate is formed. The mixture is then stirred for 3 days. After filtering, the solid is washed several times with ethanol. The surfactant is removed by extracting in a Soxhlet extractor with reflux of ethanol for 24 hours. After filtering and drying at 50° C. under vacuum, a finely divided solid is obtained.

### Example 4

### Self-Condensation of Hybrid Unit Blocks

In a 50 ml conical tube, 100 mg of hybrid unit blocks is hydrolyzed in 5 ml of hydrochloric acid solution, pH 1.5. The solution is placed for 24 hours at 25° C.

### Example 5

### Synthesis of Multifunctionalized Silica Nanoparticles (SiNP)

Several peptides can thus be grafted onto silica nanoparticles in controlled ratios at the same time.

Synthesis: Fluorescent Nanoparticles are Prepared as Follows:

In a round-bottom flask equipped with a magnetic stirrer, 150 ml of cyclohexane and 40 ml of Triton X-100® are added. Next, 20 ml of ammonia solution (4.6 mol/1) was added dropwise and the whole was stirred for 10 minutes. 40 ml of hexanol was added followed by 42 n1 of FITCSi(OEt)₃ in DMSO (0.39 mol/1) and 7.5 ml of TEOS, and stirring was continued for another 12 hours. SiNPs were precipitated by adding a large quantity of Et₂O and washed in a Soxhlet extractor using EtOH as solvent.

Hybrid peptides (1) Si(OEt)₃(CH₂)₃NHCO—βAla₄[NRP] and hybrid peptides (2) Si(OEt)₃(CH₂)₃NHCO—βAla₅c[RGD] are synthesized first by solid-phase peptide synthesis (SPPS) using the “Fmoc” strategy on trityl resin, then derivation with triethoxysilylpropylisocyanate is carried out in solution:

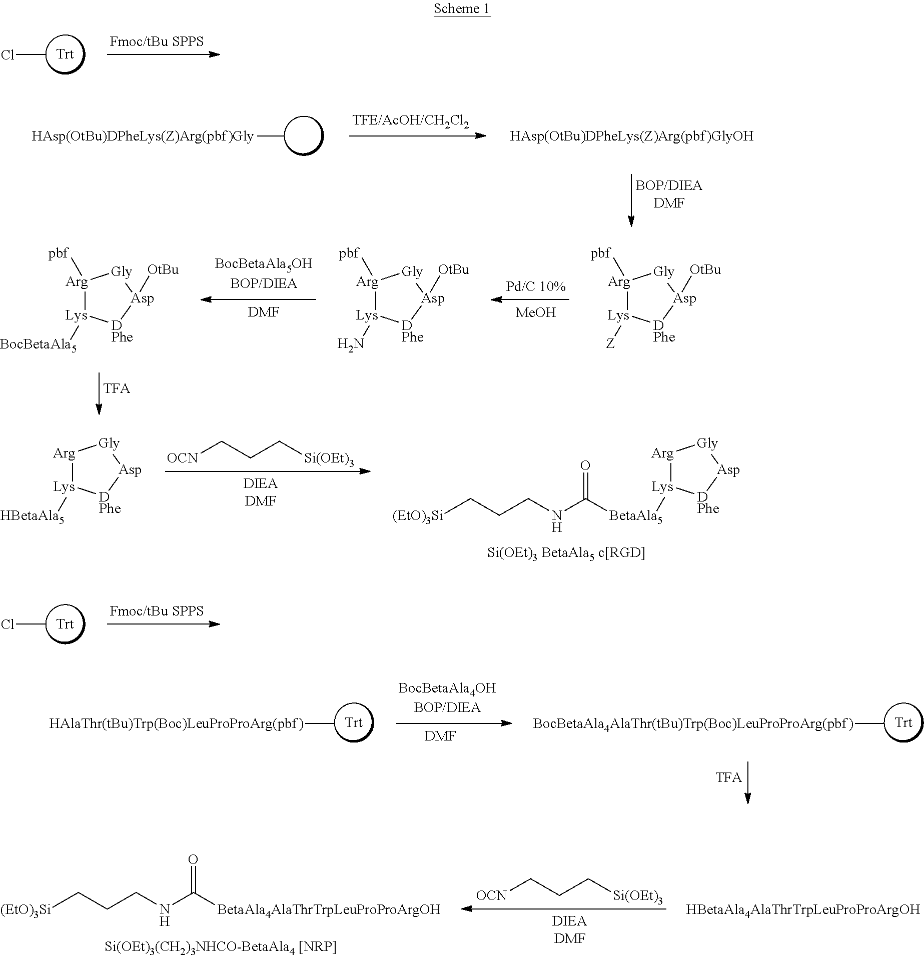

Multifunctional SiNPs are Prepared According to the Following Method:

2 ml of DMF/TFA in a 99/1 (v/v) ratio is stirred in a Falcon tube equipped with a magnetic stirrer. 100 mg of fluorescent SiNPs is added, then in equal quantities hybrid peptides (1) (1.57 mg, 1.3 pmol) and (2) (1.78 mg, 1.3 pmol).

The mixture is heated at 65° C. for 12 hours.

RP-HPLC was used to monitor the completion of the reaction by verifying the disappearance of hybrid peptides. The mixture is cooled at room temperature and centrifuged at 3,000 rpm. The product obtained is washed once with DMF and twice with dichloromethane and dried under vacuum for 12 hours.

The following elemental analysis was obtained: % N=1.74; % C=0.89

### Example 6

### Preparation of Xerogel

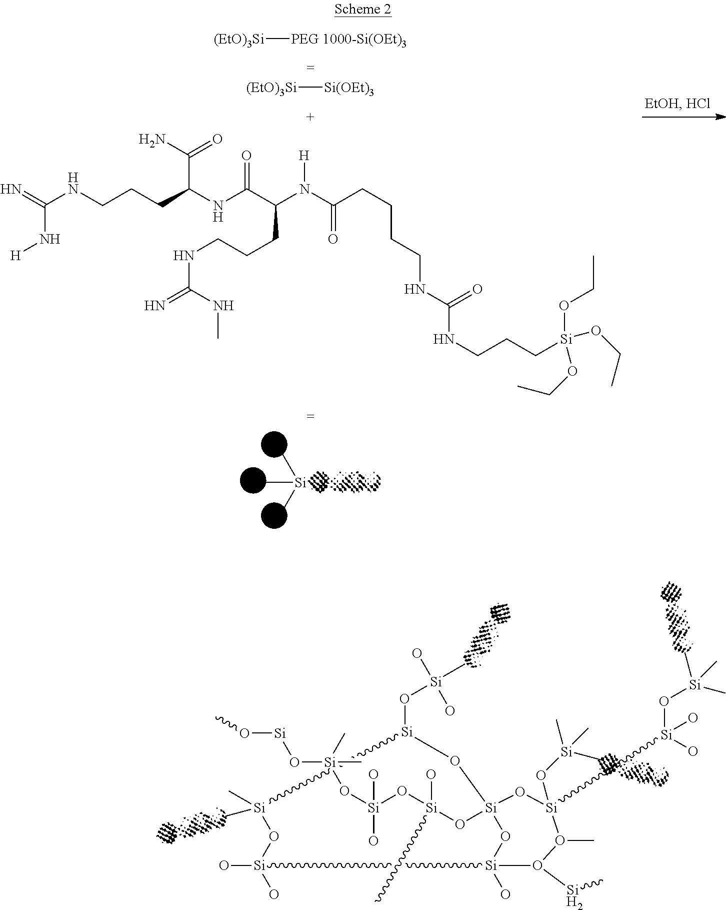

A colloidal solution containing a mixture of bis-silylated polyethylene glycol (PEG1000) and the hybrid peptide trialkoxysilyl (in this case the antimicrobial sequence Si(OEt)₃(CH₂)₃NH—CO-Ahx-Arg(Pbf)-Arg(Pbf)-NH₂) acid ethanol is prepared then poured into a Petri dish under controlled relative humidity. Evaporation of the solvent leads the inorganic polymerization process to form a three-dimensional network in the form of a flexible membrane. The membrane thus obtained is endowed with the property carried by the bioactive peptide. These membranes, whose form and size can be defined, can find application in medical devices.

Once again, this method is quite simple and fast and allows the use of mixtures of various bioactive peptides, at room temperature.

### Example 7

### Preparation of Hybrid Hollow Tubes

In the literature, this type of structure requires a 24-mer peptide linked to a lipid tail and a beta layer forming a sequence in order to induce and maintain the assembly of triple helixtype tropocollagen. According to the present invention, the use of much shorter hybrid peptides (9 amino acids) allowed the formation of irreversiblyfixed triple helices. This yields a block that forms a hollow tube.

Transmission electron microscopy (TEM) images show very regular tubes with diameters of 14 nm and wall thickness of 3.5 nm.

This type of matrix could be used as a coating on the cellular level or as an implantable collagen mimic

Synthesis of Hybrid Peptide

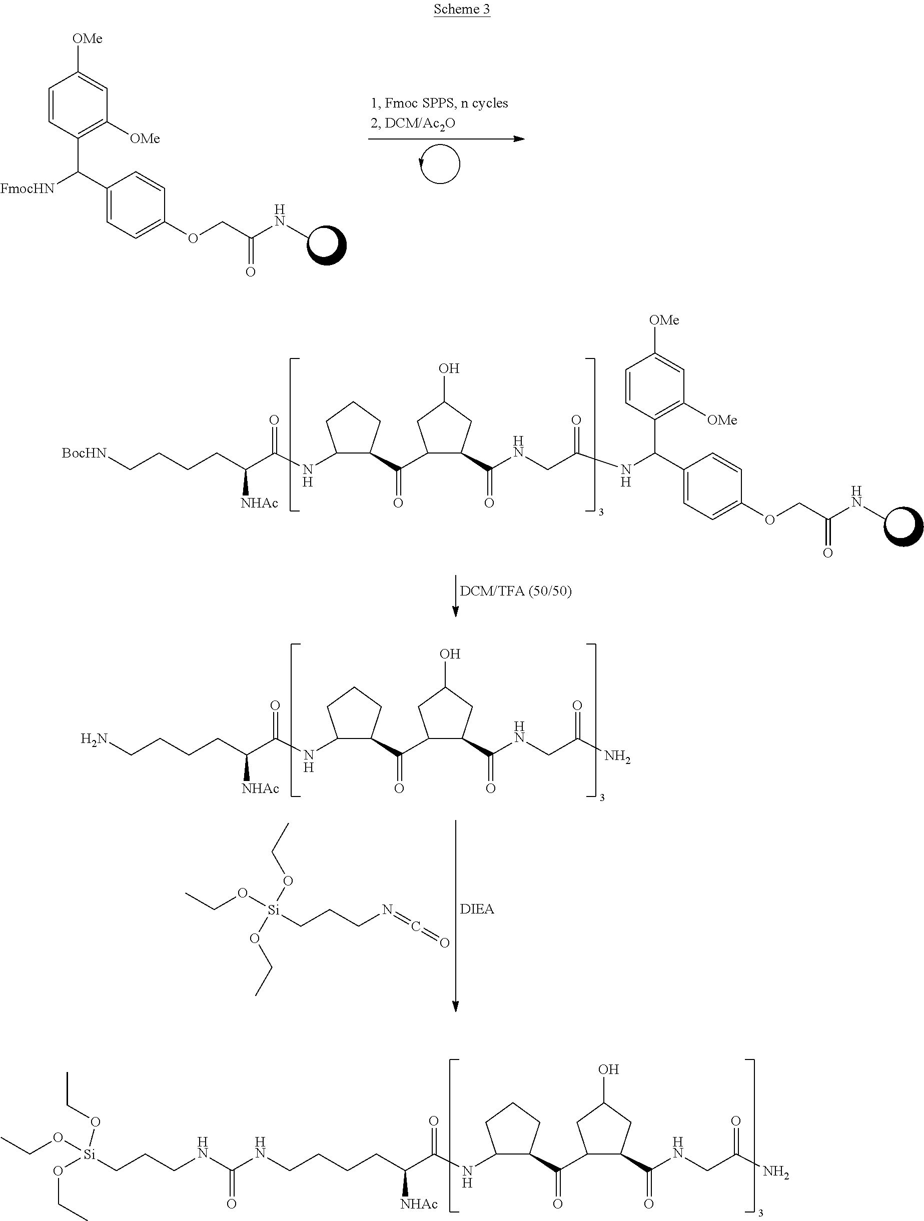

The synthesis of peptidecollagen hybrids was carried out on a solid support using “Rink” amide resin and an Fmoc/tBu strategy. The trialkoxysilylspacer fragment was grafted onto the side chain of the lysine placed at the N-terminus.

Self-Assembly:

After the hybrid peptides were synthesized, hollow tubes of peptide-silica hybrids were prepared by the method of injecting ethanol-in-water colloidal solution. The hydrolysis of trialkoxysilane into hybrid peptides was carried out in acidic (pH 4) ethanol solution at room temperature for 1 hour. This sol obtained is then injected into H₂O/EtOH solution (90/10) (pH 4, at room temperature) at a final concentration of 0.5 mg/ml⁻¹ and incubated at 45° C. for 24 hours.

### Example 8

### Synthesis of Comb-Shaped Hybrid Polymers

Presented herein is polymerization of a tripeptide (Gly-Phe-Arg) sequence in order to produce comb-shape polymers built on bis-silylated lysine scaffolding with branched peptide sequences.

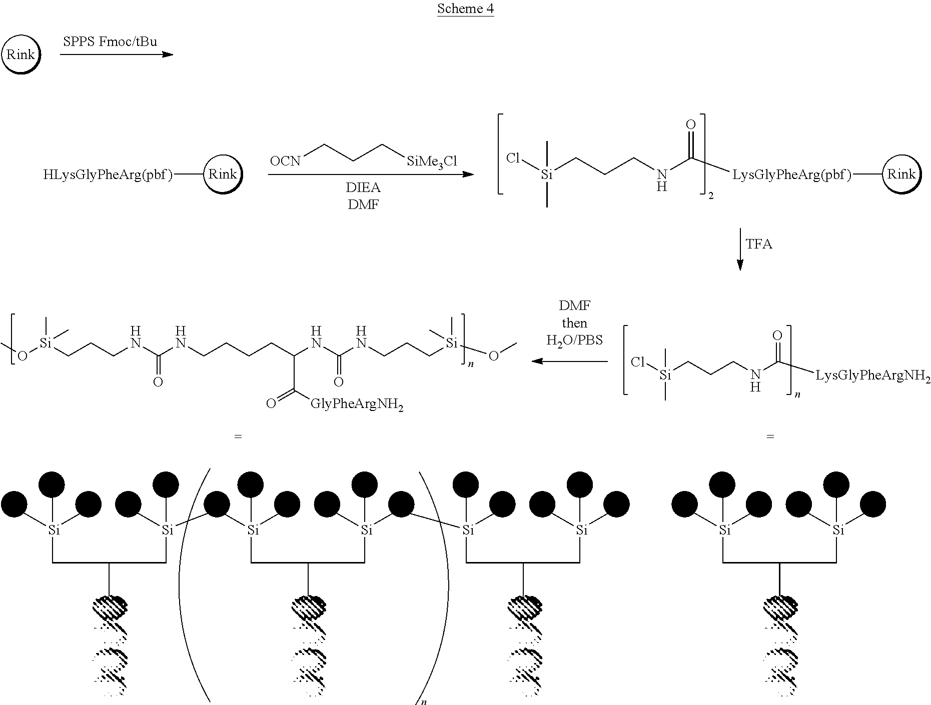

The first step is synthesis of the hybrid peptide having lysine at its N-terminus which is functionalized by isocyanatopropyldimethylchlorosilane. In this case, only one hydrolysable functional group is present on each silicon atom. Unlike the trialkoxysilyl group, each functional group can react only once, which gives non-crosslinked, i.e., linear, polymers.

The polymerization takes place in water, under neutral conditions (pH 7), at room temperature. According to peptide sequence and conditions, various lengths can be obtained. In the Scheme above, polymers of ˜5,000 g/mol are obtained by precipitating the polymerized material.

Synthesis of Hybrid Peptide:

The hybrid peptide [OHMe₂-Si(CH₂)₃NHCO₂Lys]-Phe-Gly-Arg-NH₂ is synthesized according to a standard Fmoc/tBu SPPS strategy, followed by derivatization on resin of the free ε- and α-amino groups of the N-terminal lysine by isocyanatopropyl dimethylchlorosilyl. After cleavage, the hybrid peptide is obtained as a dimethylsiloxane derivative. Such a hybrid peptide can be characterized by LC/MS. It should be noted that in the acidic aqueous solution used to prepare the sample, the intermolecular cyclized hybrid peptide is detected (m/z 806) by ESI+LC/MS. This species is in equilibrium with the linear hybrid peptide (m/z 824).

Polymerization:

In a round-bottom flask (20 ml), [OHMe₂-Si(CH₂)₃NHCO]₂Lys-Phe-Gly-Arg-NH₂ (500 mg, 0.47 mmol) in DMF (5 ml) was added with stifling. Then PBS (pH 7.4) was added dropwise until pH 7 was reached. A white precipitate appears after 30 minutes. The precipitate is filtered then characterized by steric exclusion chromatography.

A monomeric fraction (t=19′) is also detected. This can be removed by dialysis. Polymers of ˜5,000 g/mol are obtained, corresponding to chains of n=8-9 blocks.

### Example 9

### Synthesis of Comb-Shaped Hybrid Polymers Via Dichloromethylsilane Fragments

By choosing a dichloromethyl silane derivative to functionalize the N-terminus of the peptide sequence, comb-shaped silicon peptide polymers can be obtained using the same strategy as described in example 8, without requiring lysine derivative.

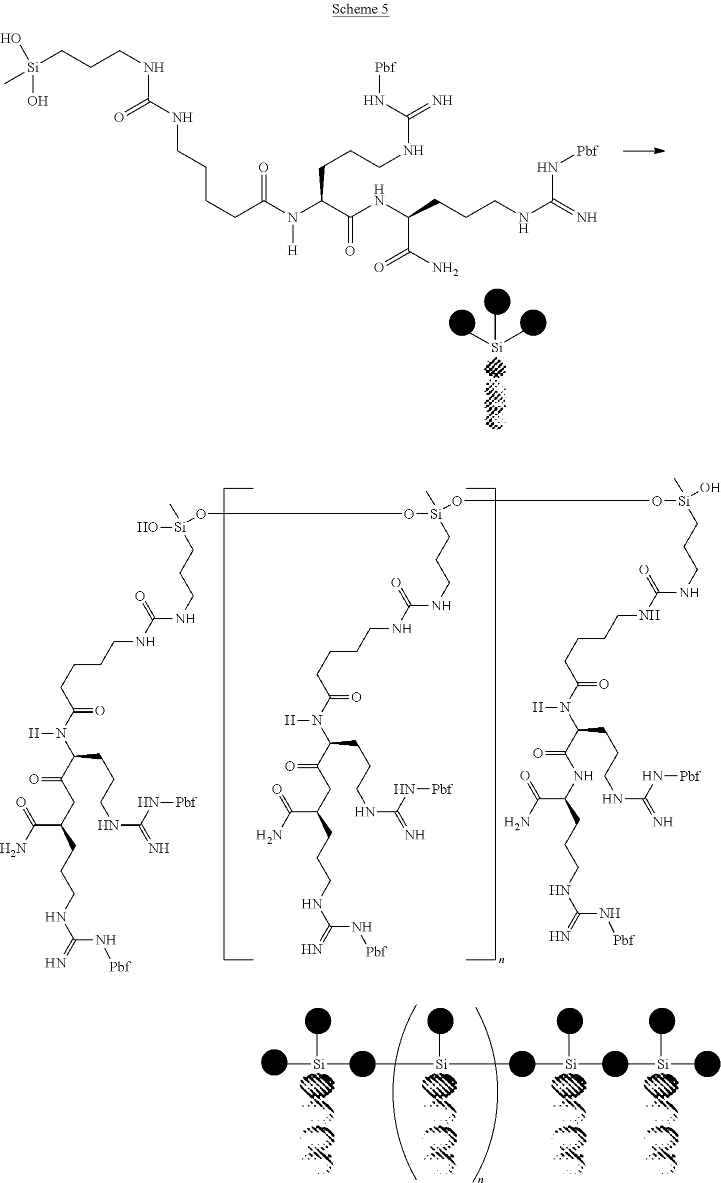

The hybrid peptide [(OH)₂MeSi—(CH₂)₃NHCO]₂Ahx-Arg(Pbf)-Arg(Pbf)-NH₂ was prepared in solution during reaction with dichloro(3-isocyanatopropyl)(methyl)silane and the protected peptide H-Ahx-Arg(Pbf)-Arg(Pbf)-NH₂. In the case of the dichloromethylsilyl derivative, two hydrolysable functional groups are present on each silicon atom. Unlike the trialkoxysilyl group, each functional group (one on each hybrid peptide) can react twice, which gives, non-crosslinked, linear comb-shaped polymers with branched peptide sequences.

The polymerization takes place in water, under neutral conditions, at room temperature.

Synthesis:

The hybrid peptide [Cl₂MeSi—(CH₂)₃NHCO]₂Arg(Pbf)-Arg(Pbf)-NH₂ is synthesized using the standard Fmoc/tBu SPPS technique on “Sieber amide” resin.

After cleavage with 1% TFA in DCM, the protected peptide H-Arg(PBF)-Arg(PBF)-NH₂ is obtained.

To the solution of H-Ahx-Arg(PBF)-Arg(PBF)—NH₂ (0.1 mmol) in 100 μl of DMF were added DIEA (2.1 eq) and dichloro(3-isocyanatopropyl)(methyl)silane (1.2 eq). The reaction mixture is left stirring for 2 hours at room temperature. After 120 minutes, the reaction was followed by HPLC.

Ether (30 ml) was poured into the reaction mixture to cause precipitation.

The precipitate was suspended in ether and recovered again.

This procedure was repeated three times to remove dichloro(3 and DIEA. All of the crude compounds were analyzed by analytical ESI+LC/MS and used without additional purification.

LC/MS indicates dimer and trimer presence showing the start of the polymerization process, even in a water/acetonitrile/TFA mixture in a proportion of 1/1/0.001.

Polymerization

In a round-bottom flask (20 ml), [OH₂MeSi—(CH₂)₃NHCO]₂-Arg(PBF)-Arg(PBF) NH₂ (500 mg) in DMF (5 ml) was added with stirring. Then water was added dropwise.

A white precipitate appears after 30 minutes. The precipitate is filtered then characterized by SEC.

A monomeric dfe fraction (t=24′) is also detected corresponding to cyclized hybrid peptide. This can be removed by dialysis. Polymers of ≈14,470 g/mol are obtained, corresponding to chains of n=30 blocks.

### Example 10

### Synthesis of a Hybrid Peptide and Application Thereof in the Preparation of Antimicrobial Materials

The following hybrid peptide was synthesized:

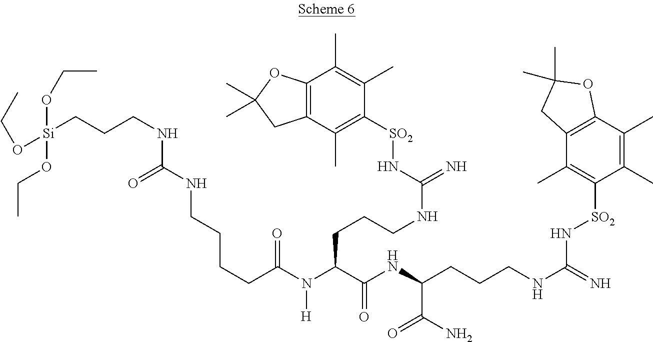 [SMILES: CCO[Si](CCCNC(=O)NCCCCC(=O)N[C@@H](CCCNC(=N)NS(=O)(=O)c1c(C)c(C)c2c(c1C)CC(C)(C)O2)C(=O)N[C@@H](CCCNC(=N)NS(=O)(=O)c1c(C)c(C)c2c(c1C)CC(C)(C)O2)C(N)=O)(OCC)OCC]

The sequence Ahx-Arg-Arg is known to have antibiotic properties.

The sequence H-Ahx-Arg(Pbf)-Arg(Pbf)-NH₂ was prepared on “Sieber amide” resin. The trialkyoxysilyl group was grafted onto the terminal amine of the peptide cleaved directly from the resin, without purifying the latter, with 3-isocyanatopropyltricthyoxysyliane (ICPTS). ICPTS is soluble in diethylether whereas the protected hybrid peptide is not, which allowed easy recovery of the desired product on a 100 mg scale.

A 60-70% yield was obtained with a degree of purity greater than 95%.

Direct Synthesis of a Thin Layer

The hybrid peptide obtained in the preceding step was dissolved with TEOS in acidic ethanol solution (pH 1.5).

After a few minutes of stirring, a clear and stable solution was obtained. This solution was deposited on a clean glass substrate by dipping. The dipped glass plates were then dried, treated with TFA/dichloromethane (1/1, v/v) solution in order to remove the protective groups, then dried again.

The surface peptide density was estimated at 1.35 peptide per nm² by a spectrophotometry technique based on the reversible complexing of Coomassie brilliant blue stain with guanidine groups.

Properties of the Thin Layer Obtained

The functionalized glass plates were incubated in Petri dishes at 37° C. in the presence of a suspension of E. coli in the exponential growth phase. These plates were washed and covered with ethidium bromide before examination under the microscope. A control sample (glass plate not treated with the hybrid peptide, but covered with TEOS) was incubated and treated under the same conditions. The comparison shows that numerous colonies are present on the glass plate without the hybrid peptide, whereas the plate with the peptide has none.

### Example 11

### Synthesis of a Hybrid Peptide and Application Thereof in the Preparation of Materials Useful in Catalysis

The following hybrid peptide was synthesized:

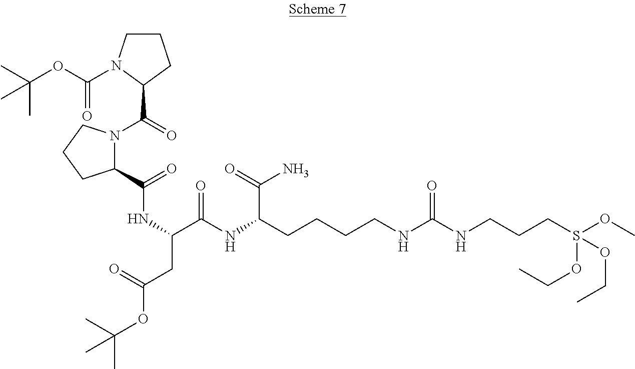 [SMILES: CCOS(CCCNC(=O)NCCCC[C@H](NC(=O)[C@H](CC(=O)OC(C)(C)C)NC(=O)[C@H]1CCCN1C(=O)[C@@H]1CCCN1C(=O)OC(C)(C)C)C(N)=O)(OC)OCC]

The sequence Boc-Pro-Pro-Asp-Lys-NH₂ is known to have catalysis properties in the aldolization reaction.

The sequence H-Pro-Pro-Asp(OtBu)-Lys-NH₂ was prepared on 2-chloro chlorotrityl resin (CTR) with synthesis beginning by anchoring lysine (Fmoc-Lys-NH₂) by its side chain to the resin. The peptide sequence was synthesized by standard coupling techniques on a solid support. The peptide was removed from the resin by mild cleavage. The trialkyoxysilyl group was grafted onto the free ε-amine of the lysine residue thus released, directly cleaved from the resin, without purifying the latter, with 3-isocyanatopropyltriethyoxysyliane (ICPTS). ICPTS is soluble in diethylether whereas the protected hybrid peptide is not, which allowed easy recovery of the desired product on a 100 mg scale.

A yield on the order of 60-70% was obtained with a degree of purity greater than 95%.

Direct Synthesis of Bioorganic-Inorganic Mesoporous Silica

The covalent inclusion of biomolecules in ordered mesoporous silica is not known a priori by direct synthesis. To date, only amino acid oligomers of random lengths have been grafted onto ordered mesoporous silicas, thus limiting the scope of their applications. Direct synthesis (“one pot” method) is an alternative for synthesizing ordered mesoporous silicas by condensation of tetraalkoxysilanes [(RO)₄Si] with organotrialkoxysilane (RO)₃SiR′ in the presence of structuring agents allowing the anchoring of organic units in the pores of the silica thus obtained. Since organic functional groups are introduced during synthesis of silica materials themselves, there is no pore-blocking phenomenon which might have appeared in the case of anchoring on a formed support. Moreover, by proceeding in this way, the organic units are divided uniformly.

Mesoporous silica with controlled pore diameters was prepared by co-hydrolysis and polycondensation of TEOS with the hybrid peptide synthesized previously (99.8/0.2 mol/mol) in the presence of Pluronic P123® (PEO₂₀POP₇₀PEO₂₀) as surfactant. The hybrid material was obtained quantitatively after the surfactant was removed by washing. The white powder obtained was treated with HMDS and TFA to remove tert-butyl and Boc groups. Moreover, HMDS made the surface of the material obtained hydrophobic since water is not recommended for the aldolization reaction. The mesoporous silica obtained has an SBA-15® ordered structure as determined by the so-called XRD technique and by measurement of N₂ adsorption-desorption. Thus, by direct comparison with “standard” SBA-15® mesoporous silica, the effective pore size obtained is not particularly different. HMDS treatment decreases surface area by 25% and pore volume by 17%, which is in the end still within an acceptable range from the viewpoint of standard SBA-15® silica.

By transmission electron microscopy, the hybrid material obtained was observed to have a highly structured hexagonal structure. The peptide load obtained was determined by elemental analysis: 16 μmol/g, i.e., 0.85% of bioorganic content, which represents quantitative inclusion in the silica matrix.

Catalysis Test

The mesoporous silica obtained previously was used as a supported catalyst (2% mol) for enantioselective aldolization in the reaction of p-nitrobenzaldehyde with acetone. The reactions were followed by HPLC. No reaction was recorded in the case of standard SBA-15® mesoporous silica. Mesoporous silica functionalized by the protected peptide also gave a negative test result. On the other hand, slow aldolization, but with a rate of 80% after 48 hours, was observed in the case of mesoporous silica functionalized by the final deprotected peptide, with stirring. Selectivity was better in DMSO (74% ee) than in acetone (48% ee). These results corroborate the liquid-phase results. Simple filtration makes it possible to separate the catalyst from the mixture, thus allowing the latter to be efficiently recycled, if need be.

1.-17. (canceled)

18. A method comprising incorporating a peptide strand A in a silica material or a metal oxide by using a peptide conjugate of the following formula (I): 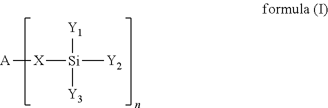
wherein:
A is a peptide fragment,
X is a spacer group,
Y₁, Y₂, Y₃, identical or different, each independently represents a hydrogen atom, a halogen atom, or an OR₂ radical wherein R₂ represents a hydrogen atom, an aryl group or a saturated or unsaturated aliphatic hydrocarbon chain comprising from 1 to 6 carbon atoms optionally substituted by an aryl, halogen or hydroxyl group,
n is an integer between 1 and 50,
wherein:
if the peptide conjugate of formula (I), wherein A is a linear peptide fragment, comprises only one Si carried by an X group on the alpha amine at the N-terminus of fragment A, then
A is a peptide fragment selected from the group consisting of an antibiotic, an antimicrobial, an antifungal, an anti-inflammatory, a catalyst, a biological receptor ligand and an enzyme inhibitor, or
Y₁ is different from Y₂ and/or Y₃
the peptide conjugate is not one of the following structures: H-YGGFLR-NH—CH₂—CH₂—CH₂—Si(OH)₃, H-YGGFLR-NH—CH₂—CH₂—CH₂—Si(OH)₂F or H-YGGFLR-NH—CH₂—CH₂—CH₂—Si(OH)F₂.

19. The method according to claim 18, wherein X is represented by a divalent radical derived from a saturated or unsaturated aliphatic hydrocarbon chain comprising from 1 to 10 carbon atoms, optionally intercalated with one or more structural linkers selected from arylene or fragments —O—, —S—, —C(═O)—, SO₂ or —N(R₁), wherein R₁ represents a hydrogen atom, an aliphatic hydrocarbon radical comprising from 1 to 6 carbon atoms, a benzyl radical or a phenethyl radical, wherein said chain is unsubstituted or is substituted by one or more radicals selected from halogen atoms, a hydroxyl group, alkyl radicals comprising from 1 to 4 carbon atoms or benzyl or phenethyl radicals.

20. The method according to claim 18, wherein peptide fragment A is a linear natural peptide strand, a linear synthetic peptide strand, a linear protected natural peptide strand, a linear protected synthetic peptide strand, a linear natural pseudopeptide strand, a linear synthetic pseudopeptide strand, a linear protected natural pseudopeptide strand or a linear protected synthetic pseudopeptide strand, or peptide fragment A comprises or consists of a cyclic natural peptide fragment, a cyclic synthetic peptide fragment, a cyclic protected natural peptide fragment, a cyclic protected synthetic peptide fragment, a cyclic natural pseudopeptide fragment, a cyclic synthetic pseudopeptide fragment, a cyclic protected natural pseudopeptide fragment or a cyclic protected synthetic pseudopeptide fragment.

21. The method according to claim 18, wherein the peptide fragment A comprises between 2 and 80 amino acids.

22. The method according to claim 18, wherein the peptide fragment A is an antibiotic, an antimicrobial, an antifungal, an antiviral, an anti-inflammatory, a catalyst, a structured peptide fragment, a biological receptor ligand or an enzyme inhibitor.

23. The method according to claim 18, wherein the Si:N ratio in moles comprised in the conjugate is between 1:0.3 and 1:100.

24. The method according to claim 18, comprising at least one of the fragments of the following formulas (II), (III) and/or (IV): 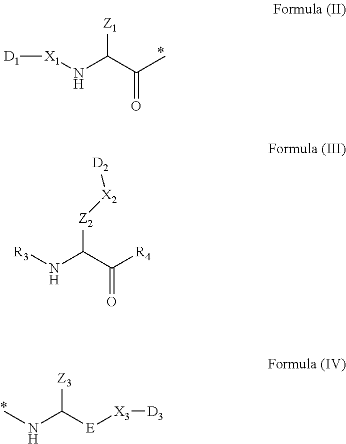
wherein,
D₁, D₂, D₃, identical or different, each independently represents a fragment of formula (V): 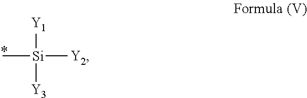
wherein, Y₁, Y₂, and Y₃ are as defined in claim 18,
X₁, X₂, X₃, identical or different, each independently represents a spacer group as defined in claim 18,
Z₁, Z₃, identical or different, each independently represents a side chain of a natural amino acid optionally substituted by a protective group,
Z₂, represent a side chain of a natural amino acid substituted by X₂ or a bond,
R₃ represents the N-terminal fragment of the peptide strand, a hydrogen atom or an N protective group,
R₄ represents the C-terminal fragment of the peptide strand, a hydrogen atom, an NH₂ group, an —OR₅ group, wherein R₅ represents a hydrogen atom or an alkyl radical of 1 to 10 carbon atoms, or a carbonyl-activating atom or group such as a halogen atom or a succinimide group,
E represents the group (C═O)— or —NH—,
* represents the at least one bond whereby the fragments are linked to the rest of the peptide conjugate.

25. The method according to claim 18 comprising the following steps:
i) activation of any one of the Y₁, Y₂, and/or Y₃ groups of the peptide conjugate defined according to claim 18,
ii) condensation, optionally in situ, of the peptide conjugate obtained according to step i) on a support material,
iii) optional rinsing step, iv) optional step of deprotecting the peptide strand.

26. The method according to claim 18, wherein the silica material is chosen in a list consisting of silica, mesoporous silica, silica nanoparticles, glass, metal oxide glass beads, silica copolymer material of peptide conjugates A with a silica precursor, or a self-condensed peptide conjugate silica copolymer material of peptide conjugates A.

27. The method according to claim 26, wherein the silica precursor is chosen from a list consisting of silicic acid, a silicate or a C₁-C₁₀ tetraalkoxysilane.

28. The method according to claim 27, wherein the C₁-C₁₀ tetraalkoxysilane is tetraethoxysilane.

29. Silica material obtainable by the method according to claim 18.

30. A method of catalyzing chemical reactions, separating products by chromatography, functionalizing nanoparticles, obtaining biocompatible matrices for the treatment of wounds or burns, obtaining material allowing facilitated electronic or ionic transport, manufacturing nanosensors, manufacturing printed circuits, preparing antimicrobial surfaces, prepating surfaces that promote cell regrowth in order to cover medical devices or silica particles used in the formulation of cosmetics comprising use of a material of claim 29.

31. The method according to claim 30, wherein the material is an incorporated peptide strand as defined in claim 18 in silica, mesoporous silica, silica nanoparticles, glass, or metal oxide.

32. The method according to claim 30, wherein the material is a silica copolymer of a peptide strand as defined in claim 18 with a silica precursor.

33. The method according to claim 32, wherein the silica precursor is silicic acid, a silicate or a C₁-C₁₀ tetraalkoxysilane.

34. The method according to claim 30, wherein the material is a self-condensed peptide conjugate silica.
